(19) United States
(12) Patent Application Publication
(10) Pub. No.: US 2008/0293670 A1
(43) Pub. Date: Nov. 27, 2008

(54) Edible tremella polysaccharide for prevention and/or improvement of intestinal disorder

(75) Inventor: Shu-Hui Yang, Kaohsiung (TW)

Correspondence Address: BACON & THOMAS, PLLC, 625 SLATERS LANE, FOURTH FLOOR, ALEXANDRIA, VA, 22314-1176, US

(73) Assignee: AGRICULTURAL RESEARCH INSTITUTE, Taichung (TW)

(21) Appl. No.: 11/802,441

(22) Filed: May 23, 2007

(51) Int. Cl. A61K 31/715; A61P 1/00

(52) U.S. Cl. 514/54

(57) ABSTRACT

It has been found that the polysaccharide isolated from a hot water extract of a Tremella mushroom without adding a chemical reagent has a novel effect of preventing and/or treating of intestinal disorder. The Tremella polysaccharide has excellent stability, water absorbing and holding ability, and swelling capacity, so it can be used to raise fecal water content and increase fecal volume, so as to improve intestinal disorder characterized by constipation and symptom caused by constipation.

### FIELD OF THE INVENTION

The present invention relates to a new use of the edible Tremella polysaccharide, and more particularly to a new use of the edible Tremella polysaccharide for preventing and/or improving of intestinal disorder.

### BACKGROUND OF THE INVENTION

Mushrooms with distinctive fruiting bodies of sufficient size to be seen with the naked eye, include about 10,000 species of varying degrees of edibility. Approximately 100 species have been tested for cultivation and only seven to eight have been cultivated on an industrial scale. The world production of cultivated edible mushrooms in 1994 was estimated to be about five million tons and was valued at about ten billion US dollars. The most popular species of cultivated edible mushrooms include Agaricus bisporus (J. Lge) Imbach, A. bitorquis (Quel.) Sacc., Lentinus edodes (Berk) Sing., Pleurotus spp., Auricularia spp., Volvariella volvacea (Fr.) Sing., Flammulina velutipes (Fr.) Sing., Tremella fuciformis Berk., Hypsizygus marmoreus (Peck) Bigel., Pholita nameko (T. Ito) S. Ito et Imai, Grifola frondosa (Dicks.: Fr.) S. F. Gray, Hericium erinaceus (Bull.: Fr.) Pers., Dictyophora indusiata (Vent.: Pers.) Fischer, Stropharia rugosoannulata Far. apud Murr., Lepista nuda (Bull.: Fr.) Cooke, and Agrocybe aegerita (Brig) Sing.

The cultivation of fruiting bodies of mushrooms deals with living organisms, for example, the mushroom itself and other microorganisms which may either be harmful or beneficial. Therefore, the methods employed in mushroom cultivation require modifications depending upon the region being cultivated, substrates available, environmental conditions and species of microorganisms encountered.

The cultivation of mushrooms for fruiting bodies production is a long-term process needing from one to several months for the first fruiting bodies to appear. Moreover, it was found that processes for extraction of polysaccharides from fruiting bodies are not considered commercially feasible, since the physicochemical properties of the products resulting from these processes were not known or regulated (U.S. Pat. No. 4,051,314). Submerged culturing of polysaccharide producers allows obtaining the end product of constant composition in a short period under controlled conditions using ecologically pure culture medium of defined composition.

Tremella mushrooms belong to the so-called jelly mushrooms, which form gelatinous fruiting bodies. The jelly mushrooms are a set of species from different taxonomical groups of Phragmobasidiomycetes, which are able to survive long periods of drought by drying to a horny texture. When moisture is again available, they absorb water and become gelatinous. This characteristic of jelly mushrooms is due to the presence of specific water absorbing polysaccharides that compose 60-70% of the dry fruiting body. Different polysaccharides have diverse characteristics. The polysaccharides, such as polygalactomannans found in the endosperm of leguminous seeds, xyloglucan from Tamarindus indica with β-1,4-glucan backbone, or β-D-glucan derived from cereal grains with a large number of glucopyranosyl units determined to linked by (1-3) and (1-4) linkages, similarly show good water absorbing capacity (U.S. Pat. No. 5,801,116, U.S. Pat. No. 6,197,318, U.S. Pat. No. 6,168,799).

Unlike the β-1-3-glucans polysaccharides from other medicinal mushrooms, jelly mushroom polysaccharides consist of other sugars as well as glucose, and therefore belong to the class of heteropolysaccharides. A unique feature of Tremella mushrooms is that their pharmacologically active polysaccharides make up most of the structural fruiting body polysaccharides while in other medicinal mushrooms pharmacologically active polysaccharides make up only a small part of the biomass. For example, in shiitake mushrooms only 31 g of lentinan was extracted from 200 kg of fresh mushrooms, Mizuno, 1999, Int. J. Medicinal Mushrooms, 1:7-27.

The main pharmacologically active substance from Tremella is the polysaccharide glucuronoxylomannan, consisting of a linear backbone of 1,3-linked alpha-D-mannose with mainly xylose and glucuronic acid in side chains. The chemical structure of Tremella glucuronoxylomannan differs among various samples of even one species, and may be in some way connected with a type of polysaccharide-based method of identification. The general proportions of xylose:glucuronic acid:mannose are given in Tremella fuciformis as 1.0:2.77:4.9; 2:1:4 in T. aurantia, and 7:1:5 in T. mesenterica. Some additional saccharides can be identified in different samples of T. fuciformis, such as glucose, fucose, xylobiose and fructose.

Generally, Tremella glucuronoxylomannan has been found in cultivating different mushroom strains, that the polysaccharides extracted from the fruiting bodied and from mycelia in pure cultures are not essentially the same, although both may be pharmacologically active. A slight difference was observed in xylose: glucuronic acid: mannose proportions in Tremella fuciformis polysaccharide from fruiting bodies (1.0:2.77:4.9) and those from pure cultures of different haploid yeast-like budding strains (1:0.8-1.3:2.1-3.5).

Naturally growing or artificially cultured fruiting bodies of Tremella mushrooms have been extensively used as a composition for dietary supplement to supply high quality protein rich in essential amino acid (U.S. Pat. No. 6,383,799), stimulator of vascular endothelial cells (U.S. Pat. No. 5,616,325), anti-allergic drug (JP 1,228,480) or skin cosmetics (JP 61,289,011, JP 63,227,512, JP 3,099,005, JP 7,033,623, JP 7,126,149, JP 9,143,024).

In the present invention, a new effect of the Tremella polysaccharide and the powder thereof on prevention and/or improvement of intestinal disorder characterized by constipation and symptom caused by constipation are found.

### SUMMARY OF THE INVENTION

According to the present invention, it has been found that the polysaccharide isolated from a hot water extract of a Tremella mushroom without adding a chemical reagent has a novel effect of improving bowel function and preventing intestinal disorder, so it can be used to prevent and/or improve constipation and the symptom caused by constipation.

The Tremella polysaccharide of the present invention is a glucuronoxylomannan, an acid heteropolysaccharide, and has a linear backbone of α-(1→3)-D-mannan, substituted with β-D-xylose, β-D-glucuronic acid and β-(1→2) D-xylobiose at C2 position of mannose residue and a molecular weight of 500,000-2,000,000 dalton.

The Tremella polysaccharide of the present invention is a colorless, transparent, odorless, tasteless, and viscous material, and has water holding capacity.

The viscosity of the Tremella polysaccharide of the present invention is stable at a temperature substantially ranged from 10□ to 80□, it is also stable substantially under pH 2-10 without shear-thinning.

The Tremella polysaccharide of the present invention has an absorbency of greater than substantially 200 ml of liquid solution per gram of the polysaccharide in powder state without syneresis when immersed in the liquid solution for substantially one hour.

According to the present invention, a method of preventing and/or improving of intestinal disorder is provided. The method comprising administering a polysaccharide to a subject suffering from intestinal disorder or in need thereof of such prevention and/or improvement, wherein the polysaccharide is isolated from water extract of Tremella mushroom without adding a chemical reagent.

In an embodiment, the intestinal disorder is characterized by constipation and symptom caused by constipation.

In an embodiment, the subject is a human and the administering is performed orally.

In an embodiment, the polysaccharide is powder and the daily effective amount is substantially in the range of 2-5 grams.

The above objects and advantages of the present invention will become more readily apparent to those ordinarily skilled in the art after reviewing the following detailed description.

### DETAILED DESCRIPTION OF THE PREFERRED EMBODIMENT

The present invention provides a new use of the Tremella polysaccharide for improving bowel function and preventing intestinal disorder characterized in constipation and symptom caused by constipation. The polysaccharide is isolated from the Tremella mushroom including but not limited to Tremella fuciformis Berk, Tremella mesenterica, Tremella aurantia, and Tremella encepuala.

In order to obtain a more natural extract of Tremella mushroom, a physical method is used to extract the active polysaccharide in the present invention without adding a chemical agent. The method is described as follows. First, a raw material of the fruiting bodies of Tremella fuciformis, Berk is rinsed with water. Subsequently, a suitable amount of water is added to the raw material, and then heated to 80-175□ to extract the polysaccharide in hot water. After the hot water extraction for about 0.5-4 hours, it is centrifuged at 3000-5000 rpm for about 3-10 minutes to obtain a polysaccharide solution, which is colorless, transparent, odorless, tasteless, and viscous. Besides, the residues can be resuspended in the water and then heated and centrifuged as the above steps to obtain more polysaccharide extracts. The extracted polysaccharide solution can be further dried by freeze-drying or other drying method to produce Tremella polysaccharide powder for convenient use. The content of Tremella polysaccharide approximately composes 80-85% of the fruiting bodies of Tremella mushrooms.

The extracted polysaccharide is glucuronoxylomannan, an acid heteropolysaccharide, having a linear backbone of α-(1→3)-D-mannan, substituted with β-D-xylose, β-D-glucuronic acid and β-(1→2) D-xylobiose at C2 position of mannose residue, and its molecular weight is 500,000-2,000,000 dalton. The foregoing described chemical structure is quite different from the homopolysaccharide extracted from Auricularia polytricha or other mushrooms, wherein the predominant structure of the homopolysaccharide is β-(1,3) D-glucan. Glucuronoxylomannan, the high-molecular substance, can be stored for a long period of time and shows good viscosity at temperature substantially ranged from 10□ to 80□. Similarly, the high-molecular substance is also stable substantially under pH 2-10 without significant shear-thinning. In other words, the Tremella polysaccharide has excellent stability against pH and temperature variation. In addition, the polysaccharide powder shows excellent water absorbing and holding ability and swelling capacity. Rehydrate 1.0 gram of Tremella polysaccharide powder with 200 ml of liquid solution, such as water, for one hour and then centrifuge at 12,000 rpm for 5 minutes, the rehydrated polysaccharide shows good water holding capacity without syneresis. That is to say, the polysaccharide powder has an absorbency of greater than about 200 ml of liquid solution per gram of the polysaccharide powder without syneresis when immersed in the liquid solution for substantially one hour. Therefore, the Tremella polysaccharide or the polysaccharide powder of the present invention provides anti-constipation and can be used as a composition for preventing and/or improving intestinal disorder, especially constipation and symptom caused by constipation, owing to the excellent water holding capacity and swelling capacity for increasing fecal water content and fecal volume.

It is an advantage of the present invention that the Tremella polysaccharide is extracted by a physical method without adding a chemical reagent, so the derived product is quite natural. Moreover, the extracted polysaccharide solution and the polysaccharide powder can be directly administered to the subject suffering from intestinal disorder or in need thereof such prevention and/or improvement without any post processing. Besides, the extracted Tremella polysaccharide and the powder thereof have excellent stability and water holding capacity. The following examples show the effects on anti-constipation of the Tremella polysaccharide according to the present invention.

### EXAMPLES

Test sample of the present invention was the extracted Tremella polysaccharide powder. Approximately 2-5 grams of polysaccharide powder with at least 300 ml of drink, such as water, was daily administered to seven voluntary subjects A-G (Table 1), wherein the subjects are humans suffering from intestinal disorder or in need thereof of such prevention and/or improvement. Test was separated into three phases, wherein phases 1-3 represent before, during, and after polysaccharide ingestion period, respectively. Bowel movement frequency and the fecal condition (including color, shape, and hardness) were also recorded at each phase.

TABLE 1
Information of seven voluntary subjects

| | subject | | | | | | |
|---|---|---|---|---|---|---|---|
| | A | B | C | D | E | F | G |
| Age | 29 | 20 | 17 | 60 | 80 | 38 | 47 |
| Gender | female | female | male | male | female | female | female |
| Carrier | office | student | stu- | office | retired | office | office |
| | worker | | dent | worker | | worker | worker |

Voluntary subjects A and B had a bowel movement once every 2-3 days at phase 1. Two to three grams of extracted Tremella polysaccharide powder with at least 300 ml drink were oral ingested daily at phase 2 for 7 consecutive days. Bowel movement frequency of voluntary subjects A and B were regulated to once a day from the second day of phase 2. But at the second day of phase 3 (two days after stop ingesting polysaccharide powder), bowel movement condition of both voluntary subject A and B returned to that of phase 1.

Voluntary subject C had a bowel movement once per day at phase 1, wherein bowel movement was stimulated by washing anus with warm water. Two grams of extracted Tremella polysaccharide powder with at least 300 ml drink were oral ingested daily at phase 2 for 7 consecutive days. From the second day of phase 2, voluntary subject C had a bowel movement spontaneously everyday. However, at the first day of phase 3, bowel movement condition returned to that of phase 1.

Voluntary subject D had a bowel movement once every 2 days with long defecation time (more than 20 minutes) at phase 1. Two grams of extracted Tremella polysaccharide powder with at least 300 ml drink were oral ingested daily at phase 2 for 7 consecutive days. At the first day of phase 2, voluntary subject D had a bowel movement spontaneously with short defecation time after 6-8 hours of polysaccharide ingestion. However, at the first day of phase 3, bowel movement condition returned to that at phase 1.

Voluntary subject E had a bowel movement once every 2-3 days with long defecation time (more than 30 minutes) at phase 1. Two grams of extracted Tremella polysaccharide powder with at least 300 ml drink were oral ingested daily at phase 2 for 7 consecutive days. Voluntary subject E had bowel movement spontaneously everyday with short defecation time from the second day of phase 2, wherein bowel movement frequency was regulated to once a day. However, at the second day of phase 3, bowel movement condition returned to that at phase 1.

Voluntary subject F could not defecate spontaneously for more than decade and bowel movement was facilitated by enema every 6-7 days. Three to five grams of Tremella polysaccharide powder with at least 300 ml of drink were oral ingested daily at phase 2 for 7 consecutive days. However, bowel movement condition was not significantly modified. After a 7-day break, 3-5 g of Tremella polysaccharide powder with 300 ml of drink was ingested again daily for another 7 consecutive days, wherein voluntary subject F had bowel movement at the second day of Tremella polysaccharide powder consumption.

Voluntary subject G could not defecate spontaneously for more than decade and bowel movement was facilitated by taking epsom salt every 3-4 days. Three to five grams of Tremella polysaccharide powder with at least 300 ml of drink were oral ingested daily at phase 2. However, subject G had no bowel movement spontaneously after ingesting polysaccharide for 3 days.

Accordingly, bowel movement of voluntary subjects A-E was facilitated and bowel movement frequency was regulated while Tremella polysaccharide powder was oral ingested. As regards voluntary subjects F and G, since enema and epsom salts are habitually used, bowel movement thereof cannot be facilitated significantly by ingesting Tremella polysaccharide. Therefore it is to be understood that Tremella polysaccharide has more significant effect on improving constipation caused by unbalance dietary, bad defecation or life habit for example but less effect on improving constipation with laxative abuse or constipation caused by organic disease.

Fecal conditions of the voluntary subjects with bowel movement significantly facilitated by consumption Tremella polysaccharide (voluntary subjects A-E) are recorded at each phase. As shown in Table 2, fecal hardness of each voluntary subject becomes softer and the fecal volume is increased at phase 2 in comparison with phases 1 and 3 owing to the good water absorbing and holding capacity and swelling capacity of Tremella polysaccharide. Since the fecal water content is raised and the fecal volume is increased, the muscular movement of the colon can be stimulated, so as to facilitate bowel movement and prevent or improve constipation.

TABLE 2
Fecal condition of each voluntary at each phase

| | Fecal Condition (color/shape/hardness) | | |
|---|---|---|---|
| subject | phase 1 | phase 2 | phase 3 |
| A | black/particle/ | gradually changing to dark | similar to phase 1 |
| | hard | yellow/strip-shape/soft | |
| B | dark brown/ | light brown-dark | similar to phase 1 |
| | flat-short/hard | yellow/strip-shape/soft | |
| C | light brown/ | dark yellow/strip-shape/soft | similar to phase 1 |
| | particle/hard | | |
| D | black/particle/ | dark yellow/strip-shape/soft | similar to phase 1 |
| | hard | | |
| E | black/particle/ | dark yellow/strip-shape/soft | similar to phase 1 |
| | hard | | |

It is known that the chemical salts, such as magnesium sulfate, are used as laxative to improve constipation. If the constipation is not improved, suppository, enema, or other chemical with much stronger laxative effect can be used to improve constipation. However, the medicament or the external stimulation can be harmful to human body for long-term use. Since the Tremella polysaccharide of the present invention is extracted by a physical method without adding a chemical reagent, and it is shown that the Tremella polysaccharide has an effect on facilitating bowel movement, increasing frequency of bowel movement and fecal volume and fecal water content, the edible Tremella polysaccharide can be administered to human or other mammals suffering from constipation or in need thereof such prevention and/or improvement, which is a new use of the edible Tremella polysaccharide.

In conclusion, the Tremella polysaccharide or the powder thereof provided by the present invention is extracted via hot water without adding any chemical reagent, so it is more natural and can be administered to human or other mammals. The Tremella polysaccharide or the powder thereof provided by the present invention has excellent water absorbing and holding ability and swelling capacity, so it can be used to prevent, improve, or treat constipation and symptom caused by constipation. In addition, the Tremella polysaccharide or the powder thereof shows good stability and can be stored for a long period of time. Therefore, the Tremella polysaccharide is also a new anti-constipation material which has good effects of preventing and improving intestinal disorder characterized by constipation.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method of preventing and/or improving of intestinal disorder, comprising administering a polysaccharide to a subject suffering from intestinal disorder or in need thereof of such prevention and/or improvement, wherein said polysaccharide is isolated from water extract of Tremella mushroom without adding a chemical reagent.

2. The method according to claim 1, wherein the intestinal disorder is characterized by constipation and symptom caused by constipation.

3. The method according to claim 1, wherein said administering is performed orally.

4. The method according to claim 1, wherein said subject is a human.

5. The method according to claim 1, wherein said polysaccharide is an acid heteropolysaccharide.

6. The method according to claim 5, wherein said acid heteropolysaccharide is a glucuronoxylomannan.

7. The method according to claim 1, wherein the structure of said polysaccharide has a linear backbone of α-(1→3)-D-mannan, substituted with β-D-xylose, β-D-glucuronic acid and β-(1→2) D-xylobiose at C2 position of mannose residue.

8. The method according to claim 1, wherein said polysaccharide has a molecular weight of 500,000-2,000,000 dalton.

9. The method according to claim 1, wherein said polysaccharide is a colorless, transparent, odorless, tasteless, and viscous material and has water holding capacity.

10. The method according to claim 1, wherein the viscosity of said polysaccharide is stable substantially under pH 2-10 without shear-thinning.

11. The method according to claim 1, wherein the viscosity of said polysaccharide is stable at temperature substantially ranged from 10□ to 80□.

12. The method according to claim 1, wherein said polysaccharide is powder and the daily effective amount is substantially in the range of 2-5 grams.

13. A polysaccharide for preventing and/or treating constipation and symptom caused by constipation, characterized in that said polysaccharide is isolated from water extract of Tremella mushroom without adding a chemical reagent, wherein said polysaccharide provides anti-constipation.

14. The polysaccharide according to claim 13 being an acid heteropolysaccharide, wherein said acid heteropolysaccharide is a glucuronoxylomannan.

15. The polysaccharide according to claim 13, wherein the structure of said polysaccharide has a linear backbone of α-(1→3)-D-mannan, substituted with β-D-xylose, β-D-glucuronic acid and β-(1→2) D-xylobiose at C2 position of mannose residue.

16. The polysaccharide according to claim 13, wherein said polysaccharide has a molecular weight of 500,000-2,000,000 dalton.

17. The polysaccharide according to claim 13, wherein said polysaccharide is a colorless, transparent, odorless, tasteless, and viscous material and has water holding capacity.

18. The polysaccharide according to claim 13, wherein the viscosity of said polysaccharide is stable substantially under pH 2-10 without shear-thinning.

19. The polysaccharide according to claim 13, wherein the viscosity of said polysaccharide is stable at a temperature substantially ranged from 10□ to 80□.

20. The polysaccharide according to claim 13 having an absorbency of greater than about 200 ml of liquid solution per gram of said polysaccharide in powder state without synthesis when immersed in said liquid solution for substantially one hour.
